(19) United States
(12) Patent Application Publication
(10) Pub. No.: US 2018/0008398 A1
(43) Pub. Date: Jan. 11, 2018

(54) DEVICE FOR ATTACHING A LIGAMENT TRANSPLANT

(71) Applicants: Rémi PHILIPPOT, SAINT PRIEST EN JAREZ (FR); Rodolphe LIMOZIN, AUREVILLE (FR); Stephane PLAWESKI, REVEL (FR); FOURNITURES HOSPITALIERES INDUSTRIE, QUIMPER (FR); FH ORTHOPEDICS, HEIMSBRUNN (FR)

(72) Inventors: Rémi PHILIPPOT, SAINT PRIEST EN JAREZ (FR); Rodolphe LIMOZIN, AUREVILLE (FR); Stephane PLAWESKI, REVEL (FR); Guillaume ROUYER, QUIMPER (FR)

(73) Assignees: FOURNITURES HOSPITIALIERES INDUSTRIE, QUIMPER (FR); Rémi PHILPPOT, SAINT PRIEST EN JAREZ (FR); Rodolphe LIMOZIN, AUREVILLE (FR); STEPHANE PLAWESKI, REVEL (FR); FH ORTHOPEDICS, HEIMSBRUNN (FR)

(21) Appl. No.: 15/545,596

(22) PCT Filed: Jan. 11, 2016
(86) PCT No.: PCT/FR2016/050044

§ 371 Date: Jul. 21, 2017

(30) Foreign Application Priority Data
Jan. 26, 2015 (FR) 15/50576

(51) Int. Cl. A61F 2/08; A61B 17/04; A61B 17/06

(52) U.S. Cl. CPC A61F 2/0811; A61B 17/0401; A61F 2002/0852; A61B 2017/06185; A61B 2017/0414; A61B 2017/0404; A61F 2002/0882; A61B 2017/0417

(57) ABSTRACT

This fastening device comprises a bearing member intended to bear against the outer cortex of a bone and provided with a passage orifice, and a flexible connecting member configured to connect the ligament transplant to the bearing member. The connecting member comprises a tubular portion configured to extend through the passage orifice and can be deformed between a first state in which the tubular portion has a first inner diameter, and a second state in which the tubular portion has a second inner diameter smaller than the first inner diameter. The connecting member also comprises a first and a second traction portions disposed respectively on either side of the tubular portion, the tubular portion being configured to be deformed towards the second state thereof when a traction is exerted on at least one of the first and second traction portions.

### CROSS REFERENCE TO RELATED APPLICATIONS

This application is a National Stage of PCT Application No. PCT/FR2016/050044 filed on Jan. 11, 2016, which claims priority to French Patent Application No. 15/50576 filed on Jan. 26, 2015, the contents each of which are incorporated herein by reference thereto.

### TECHNICAL FIELD

The present invention concerns a device for fastening a ligament transplant.

The invention applies in particular to the reconstruction of the anterior or posterior cruciate ligament of the articulation of the knee, following the accidental rupture of this ligament.

### BACKGROUND

In order to obtain a ligament transplant adapted to replace the ruptured anterior or posterior cruciate ligament, it is common to remove tendons from another part of the patient's body, in general the right inner tendon and the semi-tendinous tendon, because these samples cause no invalidation.

The ends of this ligament transplant are intended to be secured one to the femur and the other to the tibia via fastening devices.

A first known ligament transplant fastening device consists of an interference screw arranged to fasten the ligament transplant in a cylindrical bone tunnel by jamming the ligament transplant between the threads of the screw and the inner wall of the bone tunnel. Such a fastening device is likely to cause a lesion of the ligament transplant by the threads of the screw which may lead to a rupture of the ligament transplant. Furthermore, the fastening of the ligament transplant using such a fastening device is dependent on the patient's bone quality and on the production accuracy of the bone tunnel.

A second known ligament transplant fastening device consists of an endobutton arranged to bear on the outer cortex of a bone adjacent to the articulation, and comprising an opening configured for the passage of a fastening strip of textile material connected to the ligament transplant. However, the fastening strip which protrudes from the endobutton is likely to generate a painful friction on the patient's muscles.

### BRIEF SUMMARY

The present invention aims to overcome these drawbacks.

The technical problem underlying the invention consists therefore in providing a fastening device for a ligament transplant which is simple in structure and which allows an easy and reliable fastening of the ligament transplant, while limiting the risks of subsequent pain for the patient.

To this end, the present invention concerns a fastening device of a ligament transplant on a bone adjacent to an articulation, comprising:
a bearing member comprising a substantially rectangular shaped bearing part intended to bear against the outer cortex of a bone adjacent to the articulation, and an insertion part intended to be inserted into a tunnel formed in the bone adjacent to the articulation, the insertion part comprising a passage orifice, the axis of the passage orifice extending transversely to the extension direction of the bearing part, and
a flexible connecting member configured to connect the ligament transplant to the bearing member, the connecting member comprising:
   a tubular portion configured to extend through the passage orifice, the tubular portion delimiting a passage conduit and being deformable between a first state in which the tubular portion has a first inner diameter, and a second state in which the tubular portion has a second inner diameter smaller than the first inner diameter, and
   a first and a second traction portions connected to the tubular portion and disposed respectively on either side of the tubular portion, the first and second traction portions being capable of extending together at least partially in the passage conduit, the tubular portion being configured to be deformed towards the second state thereof when a traction is exerted on at least one of the first and second traction portions.

The configuration of the connecting member, and more particularly of the tubular portion, allows making, by inserting each of the first and second traction portions into a ligament loop provided at one end of the ligament transplant and in the passage conduit, two fastening loops capable of connecting the ligament transplant to the bearing member. The fastening of the ligament transplant is then carried out by exerting, in intra-articular, a traction on each of the first and second traction portions which causes a deformation of the tubular portion in the second state thereof and an immobilization of the first and second traction portions relative to the tubular portion. This results in a mechanically easy and resistant fastening of the ligament transplant, even in a bone of low mechanical quality.

Furthermore, due to the configuration of the bearing member, and in particular of the insertion portion, no fastening member likely to cause painful frictions for the patient protrudes from the side of the bearing member.

According to an embodiment of the invention, the tubular portion includes a first passage opening disposed on the side of the first traction portion and a second passage opening disposed on the side of the second traction portion, the free end of the first traction portion is shaped to be inserted into the passage conduit via the second passage opening and to come out from the passage conduit via the first passage opening, and the free end of the second traction portion is shaped to be inserted into the passage conduit via the first passage opening and to come out from the passage conduit via the second passage opening.

According to an embodiment of the invention, the connecting member is made of textile material.

According to an embodiment of the invention, the tubular portion comprises a plurality of interlaced fibers.

According to an embodiment of the invention, the connecting member is formed from a braided, woven or knitted strip.

According to an embodiment of the invention, the connecting member is formed from a braided, woven or knitted flat strip, sewn to form the tubular portion. According to another embodiment of the invention, the connecting member is formed from a braided, woven or knitted tubular strip.

According to an embodiment of the invention, the connecting member has a tensile strength of at least 1000 N.

According to an embodiment of the invention, the connecting member has a maximum elasticity comprised between 25 and 35%, for example in the range of 30%.

According to an embodiment of the invention, the passage orifice is internally delimited by a cylindrical surface.

According to an embodiment of the invention, the first and second traction portions are diametrically opposite relative to the axis of extension of the tubular portion.

According to an embodiment of the invention, the first and second traction portions are disposed substantially in the same radial orientation relative to the axis of extension of the tubular portion.

According to an embodiment of the invention, the first inner diameter of the tubular portion is substantially equal to or greater than the width of each of the first and second traction portions.

According to an embodiment of the invention, the bearing member is made of a biocompatible material. The bearing member may for example be made of metal, and in particular of titanium, titanium alloy, chromium-cobalt or else of stainless steel, or of plastics material such as of PEEK or ultra-high molecular weight polyethylene (UHMWPE). The bearing member may also be made of resorbable biocompatible material, such as PPLLA or PLGA.

According to an embodiment of the invention, the bearing part has a substantially flat bearing surface.

According to an embodiment of the invention, the insertion part has a rectangular section.

According to an embodiment of the invention, the passage orifice has an inner diameter comprised between 2 and 3 mm, and for example of about 2.5 mm.

According to an embodiment of the invention, the bearing part has a thickness of lower than 3 mm, and for example lower than 2 mm. According to an embodiment of the invention, the connecting member is elongated.

According to an embodiment of the invention, the connecting member has a length greater than 500 mm, advantageously greater than 800 mm, and for example of about 900 mm.

According to an embodiment of the invention, the connecting member is made of polyethylene, polyester, polyamide, polypropylene or of polyethylene terephthalate.

According to an embodiment of the invention, the tubular portion has a length comprised between 40 and 80 mm, advantageously comprised between 50 and 70 mm, and for example in the range of 60 mm.

According to an embodiment of the invention, the bearing part has a rectangular section.

According to an embodiment of the invention, the axis of the passage orifice extends substantially perpendicularly to the extension direction of the bearing part.

According to an embodiment of the invention, the tubular portion comprises a first end and a second end opposite the first end.

According to an embodiment of the invention, the first and second traction portions are respectively connected to the first and second ends of the tubular portion.

According to an embodiment of the invention, the first and second traction portions are fastened respectively to the first and second ends of the tubular portion.

According to an embodiment of the invention, the first passage opening is disposed at the first end of the tubular portion, and the second passage opening is disposed at the second end of the tubular portion.

According to an embodiment of the invention, the insertion part is secured to the bearing part.

According to an embodiment of the invention, the first and second traction portions are respectively extended by first and second traction wires.

According to an embodiment of the invention, the first and second traction wires have different colors. These dispositions allow differentiating the first and second traction wires and therefore facilitating the surgical operation.

According to an embodiment of the invention, the insertion part is centered relative to the bearing part, and the bearing member is symmetrical. However, according to a variant, the insertion part might be eccentric relative to the bearing part, and the bearing member might be asymmetrical.

### BRIEF DESCRIPTION OF THE DRAWINGS

In any case, the invention will be well understood using the following description with reference to the appended schematic drawing showing, by way of non-limiting examples, several embodiments of this fastening device of a ligament transplant.

FIG. 1 is a perspective view, partially sectional, of a fastening device of a ligament transplant according to the present invention.

FIG. 2 is a side view of a bearing member belonging to the fastening device of FIG. 1.

FIG. 3 is a perspective top view of the connecting member of FIG. 2.

FIG. 4 is a perspective view of a connecting member belonging to the fastening device of FIG. 1.

FIG. 5 is a sectional view of the fastening device of FIG. 1.

FIG. 6 is a sectional view of the fastening device of FIG. 1 during the connection to a ligament transplant.

FIG. 7 is a schematic view showing the femoral fastening of a ligament transplant using the fastening device of FIG. 1.

FIG. 8 is a perspective top view of a connecting member according to a variant the invention.

FIG. 9 is a perspective view of a connecting member according to a first variant.

FIG. 10 is a perspective view of a connecting member according to a second variant showing the connecting member before and after folding of the end portions of the connecting member.

FIG. 11 is a perspective view of the connecting member of FIG. 8 showing the first and second traction portions inserted into the tubular portion.

### DETAILED DESCRIPTION

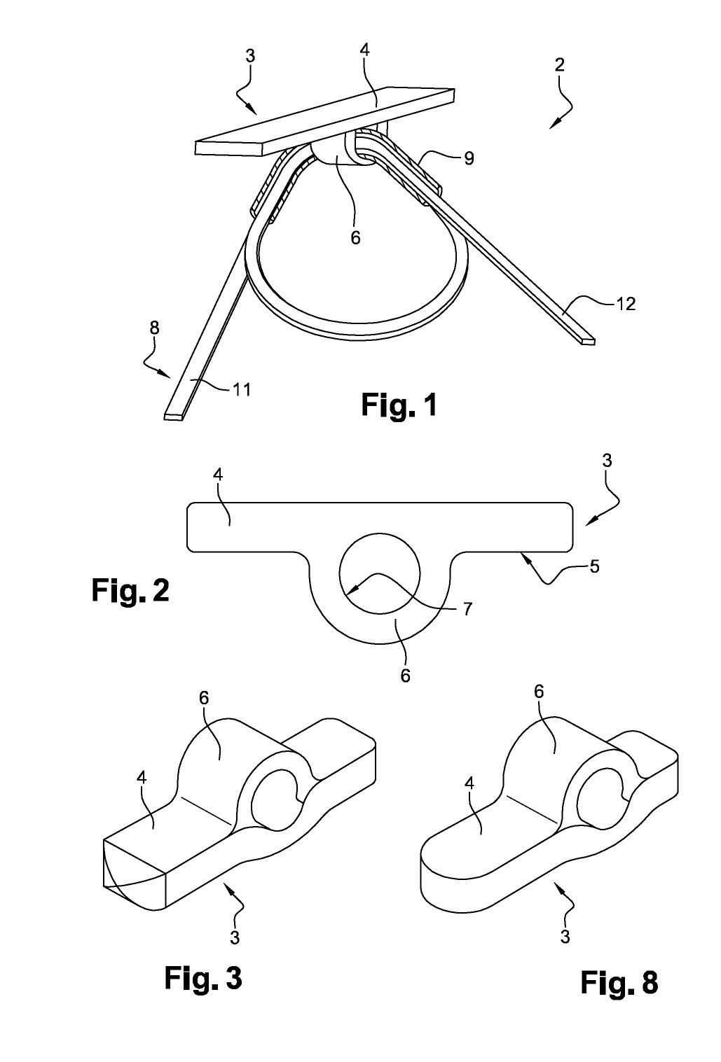

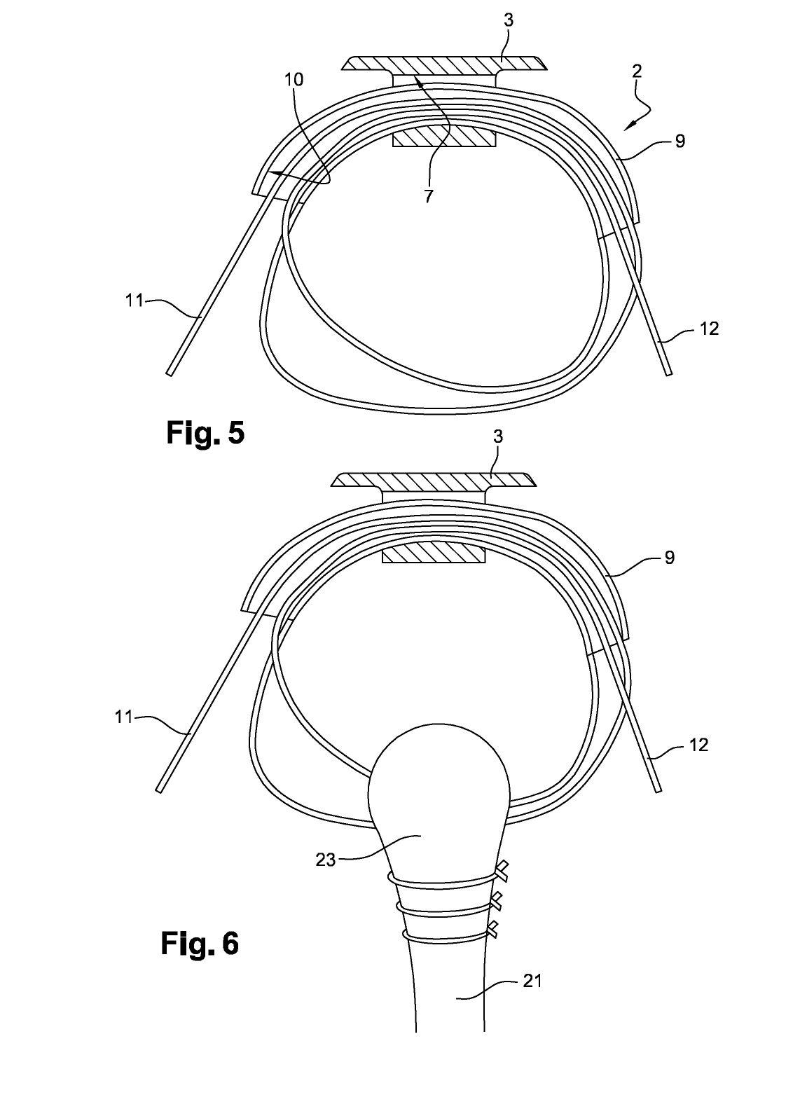

FIGS. 1 to 7 represent a fastening device 2 configured to fasten a ligament transplant in a bone tunnel formed in the end zone of a femur adjacent to the articulation of the knee. Such a fastening device 2 is more particularly used for fastening a ligament transplant ensuring the reconstruction of the anterior or posterior cruciate ligament of the articulation of the knee.

The fastening device 2 comprises a bearing member 3, also called endobutton, in one-piece part made of a biocompatible material. The bearing member 3 comprises a substantially rectangular bearing part 4 including a substantially flat bearing surface 5 intended to bear against the anterior femoral cortex. According to an embodiment of the invention, the bearing portion 4 has a length of about 12 mm, a width of about 3 mm, and a thickness lower than 2 mm.

The bearing member 3 also comprises an insertion part 6 secured to the bearing part 4 and intended to be inserted into the aforementioned bone tunnel. The insertion part 6 comprises a passage orifice 7 internally delimited by a cylindrical surface. The passage orifice 7 may have an inner diameter comprised between 2 and 3 mm, and for example of about 2.5 mm. As shown in FIGS. 2 and 3, the insertion part 6 may, for example, be centered relative to the bearing part 4, and the bearing member 3 may, for example, be symmetrical. However, as shown in FIG. 8, the insertion part 6 might be eccentric relative to the bearing part 4, and the bearing member 3 might be asymmetrical.

According to the embodiment represented in the figures, the axis of the passage orifice 7 extends transversely, and for example substantially perpendicularly, to the longitudinal direction of the bearing part 4.

The bearing member 3 may for example be metallic, and in particular made of titanium, titanium alloy, chromium-cobalt or of stainless steel. The bearing member 3 may be also made of a plastic material, such as of PEEK or ultra-high molecular weight polyethylene (UHMWPE), or of a resorbable biocompatible material, such as PPLLA or PLGA.

The fastening device 2 further comprises a flexible connecting member 8 made of synthetic textile material configured to connect the ligament transplant to the bearing member 3. The connecting member 8 may be made of polyethylene, polyester, polyamide, polypropylene or else of polyethylene terephthalate.

According to an embodiment of the invention, the connecting member 8 has a length of about 900 mm, a tensile strength of at least 1000 N and a maximum elasticity in the range of 30%.

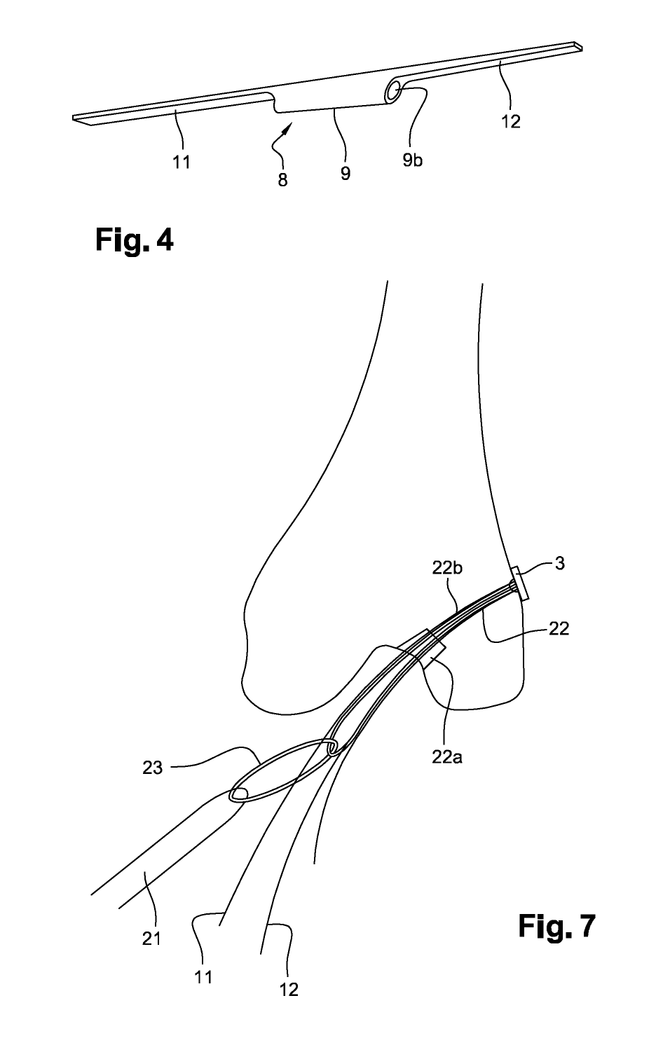

As shown more particularly in FIGS. 4 and 5, the connecting member 8 comprises a tubular central portion 9 configured to extend through the passage orifice 7 of the bearing member 3. The tubular central portion 9 delimits a passage conduit 10 and includes a first passage opening 9a disposed at a first end of the tubular central portion 9, and a second passage opening 9b disposed at a second end of the tubular central portion 9 opposite to the first end. The first and second passage openings 9a, 9b communicate with the passage conduit 10. The tubular central portion 9 may for example have a length comprised between 40 and 80 mm, and for example in the range of 60 mm.

The connecting member 8 further comprises a first traction portion 11 and a second traction portion 12 disposed respectively on either side of the tubular central portion 9 and connected respectively to the first and second ends of the tubular central portion 9. Each of the first and second traction portions 11, 12 is substantially flat and has for example a length of about 420 mm.

The tubular central portion 9 can be deformed, in the manner of a Chinese finger trap, between a first state, called relaxed state, in which the tubular central portion 9 has a first inner diameter, and a second state, called stretched state, in which the tubular central portion 9 has a second inner diameter smaller than the first inner diameter. The tubular central portion 9 is configured to be deformed towards its second state when a traction is exerted on at least one of the first and second traction portions 11, 12. Indeed, such a traction causes a decrease of the angle between the interlaced fibers forming the tubular central portion 9 at the cross points thereof, which induces a decrease of the outer circumference of the tubular central portion 9.

According to an embodiment of the invention, the first inner diameter of the tubular central portion 9 is substantially equal to or greater than the width of each of the first and second traction portions 11, 12.

As shown in FIG. 5, the first and second traction portions 11, 12 are capable of extending together at least partially in the passage conduit 10. More particularly, the free end of the first traction portion 11 is shaped to be inserted into the passage conduit 10 via the second passage opening 9b and to come out from the passage conduit 10 via the first passage opening 9a, and the free end of the second traction portion 12 is shaped to be inserted into the passage conduit 10 via the first passage opening 9a and to come out from the passage conduit 10 via the second passage opening 9b.

As shown in FIGS. 1 to 7, the connecting member 8 may be formed from a woven or knitted flat textile material strip, one part of which is sewn so as to form the tubular central portion 9. According to such an embodiment, the first and second traction portions 11, 12 may be disposed substantially in the same radial orientation relative to the axis of extension of the tubular central portion 9.

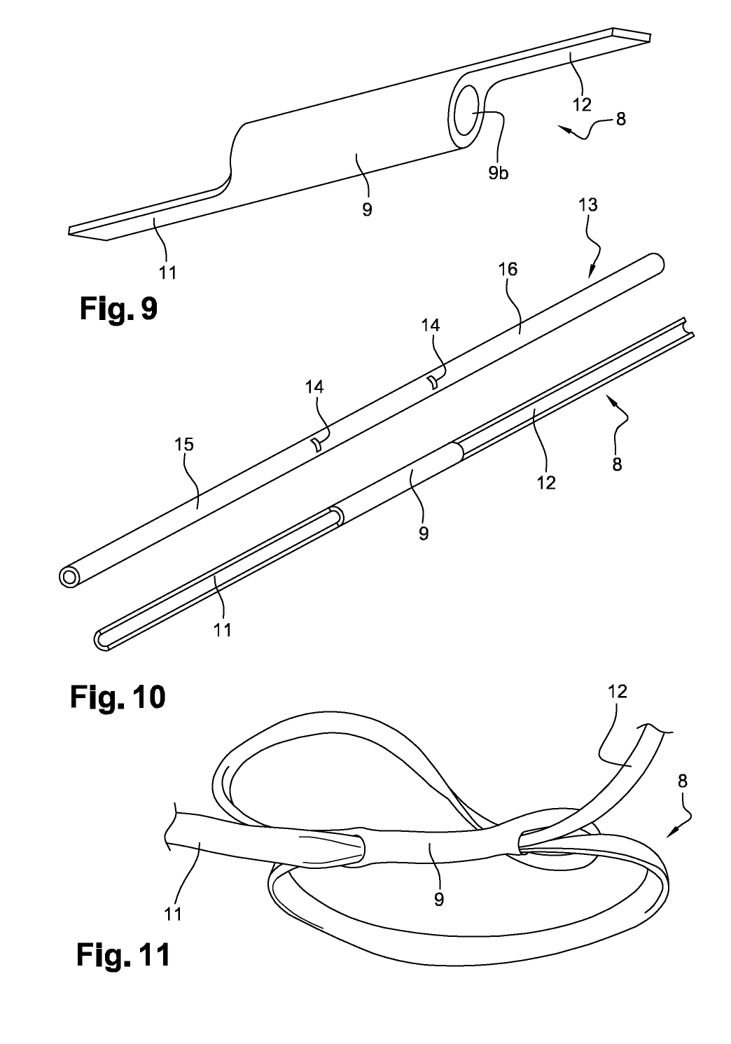

According to a first variant of the connecting member 8 shown in FIG. 9, the first and second traction portions 11, 12 may be diametrically opposite relative to the axis of extension of the tubular central portion 9.

According to a second variant of the connecting member 8 represented in FIGS. 10 and 11, the connecting member 8 may be formed from a braided, woven or knitted tubular strip 13. According to such a variant, the tubular central portion 9 is obtained by making, on the tubular strip 13, two transverse incisions 14 axially spaced of about 60 mm and of a length substantially equal to the half outer perimeter of the tubular strip, and the first and second traction portions 11, 12 are obtained by twisting or by folding, for example into eight or double eight the end portions 15, 16 of the tubular strip 13.

A femoral fastening method of a ligament transplant 21 using the fastening device 2 according to the invention will be now described.

Such a fastening method comprises in particular the following steps consisting in:
making a stepped bone tunnel 22 in the femur, such a bone tunnel 22 comprising a first tunnel portion 22a opening into an intra-articular zone matching with the natural femoral insertion zone of the cruciate ligament to be replaced and intended to receive a portion of the ligament transplant 21, and a second tunnel portion 22b opening into the femoral anterior cortex and intended for the passage of the first and second traction portions 11, 12 and for the reception of the insertion part 6, the second tunnel portion 22b having an inner diameter smaller than the inner diameter of the first tunnel portion 22a,
positioning the tubular central portion 9 of the connecting member 8 in the passage orifice 7 of the bearing member 3,
making a first fastening loop by introducing the free end of the first traction portion 11 successively into the second tunnel portion 22b, the first tunnel portion 22a, a ligament loop 23 provided at one end of the ligament transplant 21, again into the first tunnel portion 22a and the second tunnel portion 22b, and finally into the passage conduit 10 via the second passage opening 9b, and by taking the free end of the first traction portion 11 back out of the tubular central portion 9 via the first passage opening 9a,
making a second fastening loop by inserting the free end of the second traction portion 12 successively into the second tunnel portion 22b, the first tunnel portion 22a, the ligament loop 23, again into the first tunnel portion 22a and the second tunnel portion 22b, and finally into the passage conduit 10 via the first passage opening 9a, and by taking the free end the second traction portion 12 back out of the tubular central portion 9 via the second passage opening 9b,
reintroducing the free ends of the first and second traction portions 11, 12 into the bone tunnel 22 such that said ends are on the side of the intra-articular zone,
inserting the insertion part 6 into the second tunnel portion 22b and positioning the bearing surface 5 against the anterior femoral cortex,
exerting a traction on the first and second traction portions 11, 12 so as to reduce the dimensions of the fastening loops and to insert the ligament transplant 21 into the first tunnel portion 22a, and continuing to exert said traction until deforming the tubular central portion 9 in the second state thereof.

Therefore, this results in a knotless femoral fastening of the ligament transplant 21.

It should be noted that the tibial fastening of the ligament transplant 21 might be implemented by means of a second fastening device according to the invention or any other known means.

According to an embodiment of the invention not represented in the figures and in order to facilitate the operative procedure, the first and second traction portions 11, 12 might be extended respectively by a first and second traction wires of different colors.

Of course, the invention is not limited to the sole embodiments of this fastening device of a ligament transplant described above by way of examples, it encompasses on the contrary all variants.

1. A device for fastening a ligament transplant on a bone adjacent to an articulation, comprising:
a bearing member comprising a bearing part having a substantially rectangular shape and intended to bear against the outer cortex of a bone adjacent to the articulation, and an insertion part intended to be inserted into a tunnel formed in the bone adjacent to the articulation, the insertion part comprising a passage orifice, the axis of the passage orifice extending transversely to the extension direction of the bearing part, and
a flexible connecting member configured to connect the ligament transplant to the bearing member, the connecting member comprising:
a tubular portion configured to extend through the passage orifice, the tubular portion delimiting a passage conduit and can be deformed between a first state in which the tubular portion has a first inner diameter, and a second state in which the tubular portion has a second inner diameter smaller than the first inner diameter, and
a first and a second traction portions connected to the tubular portion and disposed respectively on either side of the tubular portion, the first and second traction portions being capable of extending together at least partially in the passage conduit, the tubular portion being configured to be deformed towards the second state thereof when a traction is exerted on at least one of the first and second traction portions.

2. The fastening device according to claim 1, wherein the tubular portion includes a first passage opening disposed on the side of the first traction portion and a second passage opening disposed on the side of the second traction portion, a free end of the first traction portion is shaped to be inserted into the passage conduit via the second passage opening and to come out from the passage conduit via the first passage opening, and a free end of the second traction portion is shaped to be inserted into the passage conduit via the first passage opening and to come out from the passage conduit via the second passage opening.

3. The fastening device according to claim 1, wherein the connecting member is made of textile material.

4. The fastening device according to claim 3, wherein the connecting member is formed from a braided, woven or knitted strip.

5. The fastening device according to claim 1, wherein the connecting member has a tensile strength of at least 1000 N.

6. The fastening device according to claim 1, wherein the connecting member has a maximum elasticity comprised between 25 and 35%.

7. The fastening device according to claim 1, wherein the passage orifice is internally delimited by a cylindrical surface.

8. The fastening device according to claim 1, wherein the first and second traction portions are diametrically opposite relative to the axis of extension of the tubular portion.

9. The fastening device according to claim 1, wherein the first and second traction portions are disposed substantially along a same radial orientation relative to the axis of extension of the tubular portion.

10. The fastening device according to claim 1, wherein the first inner diameter of the tubular portion is substantially equal to or greater than the width of each of the first and second traction portions.

11. The fastening device according to claim 2, wherein the connecting member is made of textile material.

12. The fastening device according to claim 11, wherein the connecting member is formed from a braided, woven or knitted strip.

13. The fastening device according to claim 12, wherein the connecting member has a tensile strength of at least 1000 N.

14. The fastening device according to claim 13, wherein the connecting member has a maximum elasticity comprised between 25 and 35%.

15. The fastening device according to claim 14, wherein the passage orifice is internally delimited by a cylindrical surface.

16. The fastening device according to claim 15, wherein the first and second traction portions are diametrically opposite relative to the axis of extension of the tubular portion.

17. The fastening device according to claim 16, wherein the first and second traction portions are disposed substantially along a same radial orientation relative to the axis of extension of the tubular portion.

18. The fastening device according to claim 17, wherein the first inner diameter of the tubular portion is substantially equal to or greater than the width of each of the first and second traction portions.

19. The fastening device according to claim 2, wherein the connecting member has a tensile strength of at least 1000 N.

20. The fastening device according to claim 2, wherein the connecting member has a maximum elasticity comprised between 25 and 35%.
